(19) United States
(12) Patent Application Publication
(10) Pub. No.: US 2014/0186435 A1
(43) Pub. Date: Jul. 3, 2014

(54) SOLUBLE TUMOR NECROSIS FACTOR RECEPTOR (STNF-R) USED AS A TARGETING AGENT TO TREAT ARTHRITIS AND OTHER DISEASES

(71) Applicants: HENRY J. SMITH, TEMECULA, CA (US); JAMES R. SMITH, LAGUNA NIGUEL, CA (US)

(72) Inventors: HENRY J. SMITH, TEMECULA, CA (US); JAMES R. SMITH, LAGUNA NIGUEL, CA (US)

(21) Appl. No.: 14/199,781

(22) Filed: Mar. 6, 2014

Related U.S. Application Data
(63) Continuation of application No. 13/507,897, filed on Aug. 6, 2012, now abandoned.
(60) Provisional application No. 61/627,820, filed on Oct. 19, 2011.

(51) Int. Cl. A61K 47/42; A61K 9/00; A61K 31/519; A61K 9/127

(52) U.S. Cl. CPC A61K 47/42; A61K 9/127; A61K 9/0019; A61K 31/519
USPC 424/450; 424/491; 514/249; 427/2.14

(57) ABSTRACT

This invention describes the use of sTNF-R as a targeting agent attached to liposomes incorporating anti-inflammatory drugs to treat arthritis and other inflammatory diseases. A variety of steroidal and non-steroidal drugs and disease modifying drugs and other anti-inflammatory compounds may be incorporated into the sTNF-R coated liposomes. The sTNF-R coated drug liposomes will accumulate within the inflamed site where the drug is released for maximum therapeutic effect. Other nanosized drug delivery vehicles such as dendrimers, micelles, nanocapsules and nanoparticles may be similarly coated with sTNF-R and used to deliver the drug to the site of inflammation.

### CROSS-REFERENCE TO RELATED APPLICATIONS

This present application is a continuation of U.S. patent application Ser. No. 13/507,897 filed on Aug. 6, 2012, which claims priority to provisional patent application No. 61/627,820 filed on Oct. 19, 2011 and titled “Soluble tumor necrosis factor receptor (sTNF-R) used as a targeting agent for anti-inflammatory drugs,” the entireties of which are incorporated by reference.

### STATEMENT REGARDING FEDERALLY SPONSORED RESEARCH

None

### BACKGROUND

Rheumatoid arthritis (RA) is an autoimmune disease that affects millions of people. One of the main signs of rheumatoid arthritis is swollen, painful joints. For mild cases of arthritis treatment usually consists of a non-steroidal drug such as aspirin or ibuprofen or naproxen. Other non-steroidal drugs include meloxicam, etodolac, nabumetone, sulidac, tolementin, diclofenac, diflunisal, indomethacin, ketoprofen, oxaprozin and piroxicam. For more severe cases steroidal drugs such as cortisone, prednisolone and methyl prednisolone are often used. In cases where there is disease progress 10n certain disease modifying drugs such as methotrexate, hydroxychloroquine, minocycline, sulfasalazine and intramuscular gold injections are often used in combination with steroids and non-steroidal drugs.

In addition to their therapeutic effect, these drugs all have a systemic effect and can cause serious side-reactions. It is desirable to have a treatment process that would be more effective upon the disease with less harmful side-effects.

One approach to ensure that the correct dosage of drug is administered and also to reduce the undesirable side-effects is to inject the drug instead of taking it orally. However, many injected drugs are detoxified by the liver and/or have undesirable side-effects. To improve the safety and efficacy of injected drugs there are various methods being developed to enclose the drug within specialized nanosized delivery vehicles such as liposomes, micelles, dendrimers, nanocapsules, nanoparticles and the like. Incorporating the drug into a specialized drug delivery vehicle alters its physicochemical makeup and changes the bioavailability and biodistribution of the drug within the body. For example, there are reports that anti-inflammatory drugs enclosed within liposomes are more efficacious than the drug given alone (van den Hoven J. M. et al., 2011; Vanniasinghe A. S. et al, 2009).

This invention teaches a method whereby the safety and efficacy of the drug can be further improved by attaching a targeting agent to the surface of the drug delivery vehicle. The targeting agent is a compound that will target the site of inflammation and cause the drug delivery vehicle to accumulate within the inflamed site where the drug is released for maximum therapeutic effect.

The novelty of this invention lies in the use of a particular targeting agent directed against a protein called “Tumor Necrosis Factor-alpha (TNF-a)”. Tumor necrosis factor-alpha is a pro- inflammatory cytokine secreted primarily by macrophages but also by a variety of cell types including lymphoid cells, mast cells, endothelial cells, cardiac myocytes, adipose tissue, fibroblasts, and neuronal tissue. TNF-a binds to Tumor Necrosis Factor Receptors (TNF-R) on certain cells causing them to respond in a particular fashion. There are two types of TNF receptors: TNF-R 1 (TNF receptor type 1; CD120a; p55/60) which is expressed in most tissues and TNF-R2 (TNF receptor type 2; CD120b; p75/80) which is found in cells of the immune system. TNF-a is a potent chemoattractant for neutrophils, and promotes the expression of adhesion molecules on endothelial cells, helping neutrophils migrate. On macrophages TNF-a stimulates phagocytosis, and production of interleukin-1 (IL-1) oxidants and the inflammatory lipid prostaglandin E2 . Patients with RA have inflamed joints in which TNF-a is produced in the lining and deeper layers of the synovium by cells of the monocyte/macrophage lineage; and it is postulated that the production of TNF-a by cells at the cartilage-pannus junction could lead to cartilage degradation in RA (Chu et al. 1991, 1992). The inflamed joint in rheumatoid arthritis is known to have increased concentrations of the pro-inflammatory cytokines TNF-a and interleukin-1 (IL-1) in the synovial fluid (Toussirot et al. 2004).

This invention teaches that it is possible to target the TNF-a present within the inflamed joint or tissue using soluble tumor necrosis factor receptor (sTNF-R) as the targeting moiety. By attaching the sTNF-R to the surface of a drug delivery vehicle such as a liposome containing an anti-inflammatory drug, it is possible to cause the liposomal drug to accumulate within the inflamed joint or tissue where the drug is released for maximum anti-inflammatory effect.

This teaching is counter-intuitive to conventional wisdom. It is well known that in the body cells communicate with each other via a large variety of biological messengers. For example, different types of cells secrete a variety of messengers such as hormones, growth factors and cytokines that circulate in the body until they reach their target cells where they will bind to their specific receptors on the target cell to induce it to respond in some manner. Under normal circumstances the messenger (ligand) is the mobile entity and the cellular receptor that it targets is the immobile entity being fixed to the cell membrane. There are numerous examples of various types of soluble ligands binding to their respective cellular receptors. For example, hormones such as estrogen will bind to estrogen receptors on breast cells; growth factors such as vascular endothelial growth factor will bind to vascular growth factor receptors on growing blood vessel cells; and cytokines such as tumor necrosis factor-alpha will bind to tumor necrosis factor receptors on macrophages and recruit them to participate in the inflammatory process.

Conventional wisdom teaches that in arthritis and other diseases where there is a pathological situation (e.g. excessive number of inflammatory cells that are secreting pro-inflammatory cytokines) then in order to obtain to obtain a therapeutic result it is necessary to either inhibit the activity of the inflammatory cells and/or prevent the secreted pro-inflammatory cytokines from recruiting other immune cells. For example, there are a number of commercially available drugs that can bind to circulating TNF-a and inhibit its pro-inflammatory action. lnfliximab is a chimeric mouse/human anti-TNF-a monoclonal antibody; adalimumab is a fully human anti-TNF-a monoclonal antibody; golimumab is another fully human anti-TNF-a monoclonal antibody; and certolizumab pegol is a pegylated Fab′ fragment of a humanized anti-TNF-a monoclonal antibody.

There is also a commercially available drug named etanercept which employs a different approach to binding circulating TNF-a. Etanercept is a recombinant fusion protein in which the binding fragment of TNF-R is joined to the Fc fragment of an immunoglobulin molecule. Upon injection into the patient etanercept will bind to the circulating TNF-a and prevent its pro-inflammatory action in arthritis and other diseases

In contrast to the examples listed above of drugs that will bind out circulating TNF-a this present invention teaches of a novel means of treating arthritis and other immune disorders by using soluble TNF-R as a targeting agent attached to the surface of a drug delivery vehicle such as liposomes in order to deliver the liposomal drug to the site of inflammation where the drug is released for maximum therapeutic effect.

The art is silent on the use of soluble tumor necrosis factor receptor (sTNF-R) as a targeting agent attached to drug liposomes in order to deliver the liposomal drug to the site of inflammation where the drug is released for maximum therapeutic effect.

### SUMMARY

This invention describes the use of sTNF-R as a targeting agent attached to liposomes incorporating anti-inflammatory drugs to treat arthritis and other inflammatory diseases. A variety of steroidal and non-steroidal drugs and disease modifying drugs and other anti-inflammatory compounds may be incorporated into the sTNF-R coated liposomes. Upon injection into the patient the sTNF-R coated drug liposomes will accumulate within the inflamed site where the drug is released for maximum therapeutic effect. Other drug delivery vehicles such as dendrimers, micelles, nanocapsules and nanoparticles may be similarly coated with sTNF-R and used to deliver the drug to the site of inflammation.

### DESCRIPTION OF INVENTION

Inflammation is the natural response of tissues to bodily injury. Clinical signs of inflammation include pain, heat, swelling, and redness at the site of the injury. Inflammation may also involve loss of function of the involved tissues. Inflammation is normally a localized, protective response following trauma or infection. However, if the agent causing the inflammation persists for a prolonged period of time, the inflammation becomes chronic. Chronic inflammation can result from a viral or microbial infection, environmental antigens, autoimmune reaction, or persistent activation of inflammatory molecules.

The inflammatory process involves a complex biological cascade of molecular and cellular signals that result in the typical clinical signs of inflammation. At the site of the injury cells release molecular signals that cause a number of changes in the affected area: dilation of blood vessels, increased blood flow, increased vascular permeability, exudation of fluids containing proteins like immunoglobulins, and invasion by leukocytes including granulocytes, monocytes, and lymphocytes that participate in the inflammatory response.

Acute inflammation is a normal process that protects and heals the body following physical injury or infection. Acute inflammation involves local dilation of blood vessels as well as increased vessel permeability to improve blood flow to the injured area. At the site of an infection or injury, mast cells, platelets, nerve endings, endothelial cells, and other resident cells release signaling molecules and chemoattractants that recruit leukocytes to the affected area. Neutrophils are the first leukocytes to appear at the injured site. These cells phagocytose and kill invading microorganisms through the release of non-specific toxins, such as superoxide radicals, hypochlorite, and hydroxyl radicals. Neutrophils also release pro-inflammatory cytokines, including interleukin-1 (IL-1), interleukin-6 (IL-6), tumor necrosis factor alpha (TNF-a) and others. These cytokines in turn induce other cells to participate in the inflammatory response.

When inflammation persists for months or years it becomes chronic inflammation. Chronic inflammation is primarily mediated by macrophages at the inflamed site. Macrophages engulf and digest microorganisms, foreign invaders, and senescent cells. Macrophages also release several different pro-inflammatory cytokines including IL-1, 1NF-a, and prostaglandins, that perpetuate and exacerbate the inflammatory response. Chronic inflammation is associated with a wide variety of diseases including asthma, Crohn's disease, rheumatoid arthritis, polymyalgia rheumatica, tendonitis, bursitis, laryngitis, gingivitis, gastritis, otitis, celiac disease, diverticulitis, and inflammatory bowel disease. Additionally, there is increasing evidence that a number of chronic diseases have inflammatory components, such as atherosclerosis, obesity, diabetes and cancer (Drake V. J. 2007)

In this invention the terms “inflammation” and “inflamed site” will include both discrete areas of inflammation and also systemic areas of inflammation. For example the arthritic joint is an example of a discrete area of inflammation; while the generalized vasculitis in systemic lupus erythematosus is an example of systemic tissue inflammation. In this invention the term “anti-inflammatory drug” will refer to all drugs that can directly or indirectly interfere with the inflammatory process including: steroidal and non-steroidal drugs, disease modifying drugs, and immune modulating drugs.

This invention teaches a method for improved delivery of pharmaceutical compounds to a site of inflammation. The target tissue may be an inflamed area within an affected joint, or tissue, or organ. The invention describes the process of incorporating anti-inflammatory drugs into nanosized drug delivery vehicles; attaching s1NF-R to the surface of the drug delivery vehicle; and administering a therapeutic dosage of the novel pharmaceutical compound to the patient with arthritis or other inflammatory condition. Upon injection into the patient the nanosized drug delivery vehicle will circulate in the blood stream until it reaches an area of inflammation where the blood vessels have enlarged endothelial pores. The nanosized drug delivery vehicle will extravasate thru the enlarged pores into the inflamed tissue. Here the sTNF-R will bind to 1NF-a secreted by cells or present in the local environment and thus become trapped within the inflamed area. Over time the drug is released from the drug delivery vehicle into the inflamed site where it will have maximum therapeutic effect.

The sTNF-R described in this invention can be prepared from either the TNF-R 1 receptor or the TNF-R2 receptor as both receptors will bind TNF-a. The TNF-R can be isolated from the cellular membrane of cells by standard laboratory techniques. For example, TNF-R bearing cells are homogenized and the cell membranes isolated by differential centrifugation. The cell membranes are solubilized using a variety of detergent solutions and the soluble receptors are then purified using gel-chromatography, or high pressure liquid phase chromatography, or other standard laboratory techniques. These methods are well known in the art and are included within the scope of this invention.

TNF-R can also be prepared as a recombinant protein using genetic engineering techniques. For example, the genetic code for TNF-R is cloned using the polymerase chain reaction and attached to plasmid DNA. The altered plasmid DNA is used to transform E. Coli bacteria which are grown in fermentation tanks. The transformed bacteria produce human TNF-R which is purified using standard methods such as ion exchange chromatography, and/or gel permeation and reverse-phase chromatography. The recombinant TNF-R may be expressed either complete, or as a fragment which has TNF binding capacity, or as part of a recombinant fusion protein. In this context, TNF-R refers to either the complete tumor necrosis factor receptor, and/or the binding fragment of TNF-R, and/or TNF-R as a component of a fusion protein molecule. The recombinant TNF-R can also be produced using other recombinant protein expression systems such as yeast cells or insect cells or mammalian cells. The methods of genetic engineering and down stream processing are well known in the art and are included within the scope of this invention.

### Nanosized Drug Delivery Vehicles.

The drug delivery vehicles that can be employed in this invention include: liposomes, micelles, dendrimers, nanocapsules and nanoparticles. Any of these drug delivery vehicles can be employed provided they can incorporate an anti-inflammatory drug and that the sTNF-R can be attached to their exterior surface. In the preferred embodiment of this invention liposomes are used as the drug delivery vehicle.

Liposomes are submicroscopic lipid vesicles. They can range in size from about 25 nm to over 1,000 nm in diameter. They are composed of a bilayer lipid membrane enclosing an aqueous center. The polar heads of the phospholipids are hydrophilic and therefore align and face the exterior surface and also the interior surface of the liposome. The hydrophobic regions (tails) of the phospholipid molecules line up opposed within the lipid membrane. Soluble drugs can be enclosed within the aqueous center of the liposome while insoluble drugs are incorporated into the lipid bilayer of the liposome.

Liposomes are prepared using a mixture of one or more of the following phospholipids: egg phosphatidylcholine (EPC), hydrogenated egg phosphatidylcholine (HEPC), soy phosphatidylcholine (SPC), hydrogenated soy phosphatidylcholine (HSPC), distearoylphosphatidylcholine (DSPC), dimyristoylphosphatidylcholine (DMPC), dipalmitoylphosphatidylcholine (DPPC), phosphatidylethanolamine (PE), phosphatidylglycerol (PG), dimyristoylphosphatidylglycerol (DMPG), phosphatidylinsitol (PI), monosialoganglioside and sphingomyelin (SPM).

To prepare the targeting liposomal drug described in this invention the lipid mixture will also include a certain quantity of derivatized vesicle forming lipids such as poly(ethyleneglycol)-derivatized distearoylphosphatidylethanolamine (PEG-DSPE), and/or poly(ethyleneglycol)-derivatized distearoylphosphatidylethanolamine with a maleimide site (MAL-PEG-DSPE). The PEG moiety used is a polymer with a MW typically in excess of 2,000 daltons. Typically, a certain amount of cholesterol is included to improve the physicochemical characteristics of the liposome.

The lipid mixture is dissolved in an organic solvent and then dried to form a lipid film. The dried lipid film is then hydrated with a solution of the anti-inflammatory drug whereupon a certain portion of the drug solution will become encapsulated within the interior of the liposomes thus formed. After removal of the unentrapped free drug using column chromatography or dialysis, the drug liposomes are sized by extruding them thru orifices of decreasing pore size using a commercial extruder. This will result in unilamella drug liposomes with a standardized uniform diameter. The size of the drug liposomes to be used is critical in order to obtain the best results. Liposomes that are less than 50 nm in diameter will enclose a small amount of drug, while liposomes that are larger than 400 nm in diameter will be too large to extravasate thru the endothelial pores of inflamed blood vessels to enter the inflamed site to deliver the drug there (Maeda H. 2001). The larger liposomes are also more likely to become trapped and degraded by the liver, and to also be recognized and removed by the reticuloendothelial system (RES) of the patient. In this invention the preferred diameter of the drug liposomes will be selected to be of a standardized diameter between 50 nm and 200 nm, and more preferably between 50 nm and 120 nm and most preferably to be about 100 nm in diameter.

An alternative method of encapsulating soluble drugs is to load the drug into preformed liposomes using a pH gradient method where the aqueous interior of the liposome has a lower pH than the external medium surrounding the liposome. Amphipathic drugs will migrate and concentrate within the liposome (Hu et al. 2010). Another method of loading soluble drugs into the interior of liposomes employs an ammonium sulphate gradient method (Bolotin et al 2007). There are many different methods of loading drugs into liposomes that are known in the art and are within the scope of this invention.

Anti-inflammatory drugs that are insoluble can be incorporated into liposomes by dissolving them in an alcohol/organic solvent and co-dissolving them with the lipid mixture. The drug/lipid solution is then dried to form a lipid film. The lipid film is then hydrated in a suitable solution such as a sucrose solution or a known buffer solution. The liposomes thus formed will have the drug incorporated within the bilayer lipid membrane of the liposome. The drug liposomes are then sized by extruding them thru orifices of decreasing pore size using a commercial extruder. This will result in unilamella drug liposomes with a uniform diameter preferably in the 100 nm range. The methods of preparing liposomes are well known in the art and are included within the scope of this invention.

Liposomal drugs prepared in this manner will have the DSPE portions of the PEG-DSPE and MAL-PEG-DSPE molecules incorporated into the lipid layer, leaving the distal PEG and MAL-PEG ends free in the external environment. The sTNF-R can be attached to the maleimide site on the MAL-PEG-DSPE molecule thru a thiol link. Alternatively, a DSPE-PEG-NH 2 or DSPE-PEG-COOH molecule may be used to attach the sTNF-R to the liposome. These and other means of linking a protein to an activated PEG molecule using other linkers are well known in the art and are included within the scope of this invention (Blume G. et al. 1993).

An alternative method of attaching the sTNF-R to the surface of the liposomes is to use the post-insertion method (Allen T. M et al. 2002). In this method the drug liposomes are prepared as before but with the MAL-PEG-DSPE omitted. The sTNF-R is attached to the MAL-PEG-DSPE separately. The drug liposomes are then incubated with the sTNF-R-PEG-DSPE at a temperature above the transition temperature to allow the DSPE end of the MAL-PEG-DSPE molecule to interpose within the lipid layer of the liposome thus attaching the sTNF-R-PEG- DSPE to the surface of the lipo some.

As the above examples demonstrate there are many different methods and formulations of preparing liposomal drugs and the means by which sTNF-R can be attached to their surface. These methods are well known in the art and are included within the scope of this invention (Hansen C. B. et al 1995).

It will also be obvious to those of skill in the art that other nanosized drug delivery vehicles can be substituted instead ofliposomes and that attaching the sTNF-R to their surface will enable them to target the site of inflammation in like manner. These other drug delivery vehicles include micelles, dendrimers, nanocapsules and nanoparticles. The methods of preparing micelles, dendrimers, nanocapsules and nanoparticles are well known in the art and are included within the scope of this invention (Torchilin V. P. 2007, Jain K. K. 2005). The methods of attaching a targeting moiety to their surface are also well known in the art (Park J. W. et al. 1997; 2002) and are included within the scope of this invention.

The art is silent on the on the use of sTNF-R as a targeting agent attached to the surface of liposomes and/or other drug delivery vehicles such as micelles, dendrimers, nanocapsules and nanoparticles to deliver anti-inflammatory drugs to the site of inflammation.

The list of anti-inflammatory drugs that can be incorporated into the sTNF-R liposomes or other drug delivery vehicles include: cortisone, hydrocortisone, prednisolone, methyl prednisolone, methotrexate, hydroxychloroquine, leflunomide, minocycline, sulfasalazine, colchicine, cyclophosphamide, azathioprine, cyclosporine-A, and d-penicillamine. All these drugs can be encapsulated or incorporated into sTNF-R coated liposomes or other drug delivery vehicles and used to treat arthritis and other inflammatory diseases.

### DRUG ADMINISTRATION

A therapeutic dosage of the sTNF-R coated drug liposomes can be administered by intravenous injection, subcutaneous injection, or by direct injection into the inflamed area such as into the synovial space of the inflamed joint. When administered by intravenous or subcutaneous injection the quantity of sTNF-R present on the liposomes will be sufficient to bind out any circulating TNF-a and still retain an excess of sTNF-R liposomes. These will be available to infiltrate into the inflamed tissue and to bind to the TNF-a there thus anchoring the liposomal drug within the inflamed area. Over time the anti-inflammatory drug is released within the inflamed site where it will be most effective.

### DISCUSSION

There are a growing number of reports on the use of liposomal anti-inflammatory drugs to treat arthritis and other inflammatory diseases. For example, Metselaar J. M. et al. reported the remission of experimental arthritis by joint targeting of glucocorticoids with long-circulating liposomes (Metselaar et al 2003, 2004); Van den Hoven et al. reported that glucocorticoids encapsulated within small liposomes showed improved anti-inflammatory effects compared to the free drug on adjuvant-induced arthritis in rats (Van den Hoven et al. 2011); and Hofkens et al. similarly reported that long circulating liposomes encapsulating prednisolone phosphate strongly suppressed knee joint swelling in adjuvant-induced arthritis in mice (Hofkens et al. 2011). Koning et al. describe targeting angiogenic endothelial cells at the site of inflammation using dexamethasone phosphate encapsulated within liposomes coated with RGD peptide. The researchers found superior binding of the RGD-peptide liposomes to the inflamed site and strong anti-inflammatory effects upon the course of experimental arthritis in rats (Koning et al 2006). The use of RGD-peptide to target protein markers expressed on endothelial cells is consistent with conventional wisdom which is to use liposomal drugs coated with a targeting ligand that will bind to cellular receptors. It is of note however, that there are no prior teachings of the use of liposomal drugs coated with a soluble targeting receptor to bind to free ligands present in areas of inflammation.

This invention teaches a novel means of treating arthritis and other immune disorders using sTNF-R as a targeting agent to deliver anti-inflammatory drugs to the site of inflammation. The anti-inflammatory drug is incorporated into a liposomal formulation coated with PEG polymers. A certain fraction of the PEG polymers contain an active malemide site to which the sTNF-R is attached thus anchoring the sTNF-R to the surface of the liposome. There are many advantages to the particular composition of the compound pharmaceutical described in this invention. For example, enclosing the anti-inflammatory drug within PEG coated liposomes protects them from being degraded by the liver (first pass effect) or removed by the RES. Therefore more of the drug is bioavailable for a longer period of time. Making the drug liposomes to be a certain size (e.g. 100 nm) prevents them from extravasating thru normal blood vessels and entering into normal tissues to cause harm. However, the drug liposomes being smaller than the enlarged endothelial pores of inflamed blood vessels will extravasate thru the enlarged pores and penetrate into the inflamed tissues. Here the sTNF-R on the liposomes will bind to the TNF-a present in the inflamed site and anchor the drug liposomes in that location. Over time the drug is released from the liposome into the inflamed site where it will have the most therapeutic effect.

An important side-benefit of using sTNF-R as the targeting agent on the liposome is that it will have a direct anti-inflammatory effect of its own, distinct from the therapeutic action of the small molecule anti-inflammatory drug incorporated in the liposome. Patients with arthritis and other inflammatory diseases produce TNF-a which is present in the blood. There are a number of commercially available drugs such as: Infliximab, adalimumab, golimumab, certolizumab and etanercept that can bind to circulating TNF-a and inhibit its pro-inflammatory action. There is no teaching however, in any reports or publications, that sTNF-R can be attached to a nanosized drug delivery vehicle and used as a targeting moiety to deliver small molecule anti-inflammatory drugs to the inflamed site.

In this invention sTNF-R is used as the targeting moiety to deliver an anti-inflammatory drug delivery vehicle to the inflamed site, with the additional benefit that it may also have some therapeutic effect in its own right by binding to circulating TNF-a. For example, upon intravenous administration of the sTNF-R coated drug delivery vehicle the sTNF-R moiety will bind to any circulating TNF-a present in the blood and thus prevent its pro-inflammatory action in exacerbating systemic disease activity. Tue remaining active sTNF-R coated drug delivery vehicles will exit thru the inflamed capillaries and into the inflamed tissues and joints. Here the sTNF-R will bind to the local TNF-a being secreted by the inflammatory cells and will inhibit them from exacerbating a local inflammatory response. At the same time the sTNF-R coated drug vehicles will become anchored within the inflamed site and will accumulate there. Over time the drug is released from the delivery vehicles within the inflamed site, where it will have the best inhibitory effect upon the local pro-inflammatory cells.

The sTNF-R based pharmaceuticals described in this invention can be used to treat a wide variety of diseases that have an inflammatory component such as rheumatoid arthritis, polyarticular juvenile idiopathic arthritis, psoriatic arthritis, ankylosing spondylitis, plaque psoriasis, polymyalgia rheumatica, asthma, Crohn's disease, tendonitis, bursitis, laryngitis, gingivitis, gastritis, otitis, celiac disease, diverticulitis, and inflammatory bowel disease. They may also be used to treat osteoarthritis because although osteoarthritis is not generally considered to be an autoimmune disease there is growing evidence that the osteoarthritic joint may exhibit signs of inflammation and therefore anti-inflammatory drug therapies deserve further investigation (Walsh D. A. et al 2003, Furuzawa-Carballeda J. et al. 2008). Other examples of diseases that have an inflammatory component include systemic lupus erythematosus (SLE) where a significant number of patients have vasculitis; patients with gout where the affected joint is inflamed (Cronstein B. N. and Terkeltaub R. 2006); patients with cardiomyopathy who show signs of an inflammatory condition in the heart; and organ transplant patients experiencing rejection of the transplanted organ that exhibit inflammation at the site of graft rejection.

Additionally, there is increasing evidence that a number of chronic diseases such as atherosclerosis, obesity and diabetes have inflammatory components that may respond to treatment with anti-inflammatory drugs. These chronic diseases may also be candidates for treatment with the sTNF-R coated drug delivery vehicles carrying anti-inflammatory compounds described in this invention.

Many autoimmune diseases such as rheumatoid arthritis and SLE are systemic in nature. In addition to the inflamed joints in RA other tissues may also be inflamed. Administration of the sTNF-R drug delivery vehicles may have in addition to their therapeutic action on the discrete inflamed tissue site a more general beneficial effect upon all the inflamed areas in the body.

### EXAMPLE 1

### sTNF-R Coated Liposomes Incorporating an Anti-Inflammatory Drug

The following is an example for illustrative purposes only of a preparation of stabilized sTNF-R coated liposomes incorporating the disease modifying drug -methotrexate. The lipid mixture is typically composed SPC or HSPC, or a mixture of the two. In this example the lipid mixture is formulated as HSPC: Cholesterol: PEG2000-DSPE: MAL-PEG₂₀₀₀-DSPE using molar ratios of 2/110.06/0.01. The lipid components are mixed together in a round bottomed flask and dissolved in a chloroform/alcohol solution. Typically, there is approx. 25 mg lipid/ml organic solvent. The flask is attached to a rotary vacuum evaporator and thoroughly dried under vacuum at room temperature overnight. The dried lipid film is hydrated with a solution of methotrexate maintained at 60° C. and sonicated to prepare liposomes thus encapsulating the drug within the aqueous interior of the liposome. The drug liposomes are then extruded using a commercial extruder thru graduated membranes of decreasing pore size from 500 nm to 100 nm. This results in unilamella liposomes having a controlled diameter of about 100 nm. The process is maintained at 60° C. throughout. The liposomes are then cooled to room temperature and separated from unencapsulated free drug using column chromatography or dialysis. The drug liposomes are then mixed with the sTNF-R to allow it to attach to the MAL-PEG2000-DSPE on the surface of the liposomes. The liposomes are then purified using column chromatography to remove any remaining unbound sTNF-R. They are stored at 4° C. or lyophilized with a cryoprotectant and kept at −20° C. for longer term storage. Lyophilized liposomes are reconstituted to original volume using distilled water or physiological solution suitable for injection or infusion before use.

This example is provided for illustration and not of limitation. It will be obvious to those of skill in the art that a large variety of anti-inflammatory drugs can be encapsulated or incorporated into liposomes in like manner using known methods. It will also be obvious that the composition of the liposomes can be varied without departing from the spirit and scope of this invention which is the use of sTNF-R as the targeting moiety for a wide variety of liposomal drugs. It will also be obvious to those of skill in the art that other nanosized drug delivery vehicles such as micelles, dendrimers, nanocapsules and nanoparticles can be substituted for liposomes using known methods without departing from the spirit and scope of this invention, which is the use of sTNF-R as the targeting moiety for said drug delivery vehicles.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Bach group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

Allen T. M. et al. Cell. Mol. Biol. Lett. 2002. Vol 7 (2):218-219.
Biolotin E. M. et al. 2007. Patent EP1105098.
Blume G. et al. 1993. Biochim Biophys Acta. 1993; 1149: 180-184.
Chu C. Q. et al. 1991. Arthritis Rheum. 34(9):1125 1132.
Chu C. Q. et al. 1992., Br J Rheumatol. 31(10):653-661.
Cronstein B. C. and TerkeJtaub R. 2006. Arthritis Res.Therapy.8 (Supp 1):S3 doi:10.1 186/ar1908
Drake V. J. 2007. Research Newsletter. Linus Pauling Institute, Oregon State University.
Etanercept(Enbre1R). Immunex Corporation 1998-2011. www:amgen.com
Furuzawa-Carballeda J. et al. 2008. Clin Exp Rheumatol. 26(4):554-560.
Hansen C. B. et al 1995. Biochim Biophys Acta. 1995; 1239:133-144
Hofkens W. et al 2011. Journal of Controlled Release 152:363-369.
Hu N. et al. 2010. US Patent Application 20100119590.
Jain K. K. 2005. Technology in Cancer Research and Treatment 4(4):407-416.
Koning G. A. et al. 2006. Arthritis Rheum. 54(4) 1198-1208.
Maeda H. 2001. Adv Enzyme Regul. 2001; 41:189-207.
Metselaar J. M. et al. 2003. Arthritis Rheum. 7: 2059-2066.
Metselaar J. M. et al. 2004. Ann Rheum. Dis. 4:348-353.
Park J. W. et al. 1997. Adv Pharmacol. 1997; 40:399-435
Park J. W. et a12002. Clin Cancer Res. 2002; 8: 1172-81.
Torchilin V. P. AAPS Journal 2007, 9(2): E128-E147
Toussirot et al. 2004. Expert Opinion in Pharmacotherapy, 5(3):581-594.
Vanniasinghe A, S. et aJ. Seminars in Arthritis and Rheumatism 2009, 39(3):182-1 96. Van den Hoven J. M. et al. 2011. Mol.Pharmaceutics, 8(4):102-1015
Walsh D. A. et al 2003? August issue Arthritis and Rheumatism

What is claimed is:

1. A pharmaceutical composition comprising at least one anti-inflammatory drug encapsulated within a nanosized drug delivery vehicle, wherein a soluble tumor necrosis factor receptor (sTNF-R) is attached to an exterior surface of the nanosized drug delivery vehicle.

2. The pharmaceutical composition of claim 1, wherein the at least one anti-inflammatory drug is selected from the group consisting of steroidal and non-steroidal drugs, disease modifying drugs, and immune modulating drugs.

3. The pharmaceutical composition of claim 1, wherein the at least one anti-inflammatory drug is selected from the group consisting of cortisone, hydrocortisone, prednisolone, methyl prednisolone, methotrexate, hydroxychloroquine, leflunomide, minocycline, sulfasalazine, colchicine, cyclophosphamide, azathioprine, cyclosporine-A, and d-penicillamine, aspirin, ibuprofen, naproxen, meloxicam, etodolac, nabumetone, sulidac, tolementin, diclofenac, diflunisal, indomethacin, ketoprofen, oxaprozin, and piroxicam.

4. The pharmaceutical composition of claim 1, wherein the nanosized drug delivery vehicle is selected from the group consisting of liposomes, micelles, dendrimers, nanocapsules, and nanoparticles.

5. The pharmaceutical composition of claim 4, wherein the nanosized drug delivery vehicle is a stabilized liposome.

6. The pharmaceutical composition of claim 5, wherein the liposome has a diameter between about 50 nm and about 400 nm.

7. The pharmaceutical composition of claim 6, wherein the liposome has a diameter between about 50 nm and about 200 nm.

8. The pharmaceutical composition of claim 7, wherein the liposome has a diameter between about 50 nm and about 120 nm.

9. The pharmaceutical composition of claim 8, wherein the liposome has a diameter about 100 nm.

10. The pharmaceutical composition of claim 5, wherein the stabilized liposome has polyethylene glycol polymers (PEG) attached to the exterior surface of the liposome, and the sTNF-R is chemically linked to an active site on a distal free end of the PEG such that the attached sTNF-R is capable of binding to TNF-a.

11. A method of forming an anti-inflammatory pharmaceutical composition, said method comprising the steps:
a) encapsulating at least one anti-inflammatory drug in a nanosized drug delivery vehicle; and
b) attaching a soluble tumor necrosis factor receptor (sTNF-R) to an exterior surface of the nanosized drug delivery vehicle.

12. The method of claim 11, wherein the at least one anti-inflammatory drug encapsulated in step a) is selected from the group consisting of cortisone, hydrocortisone, prednisolone, methyl prednisolone, methotrexate, hydroxychloroquine, leflunomide, minocycline, sulfasalazine, colchicine, cyclophosphamide, azathioprine, cyclosporine-A, and d-penicillamine, aspirin, ibuprofen, naproxen, meloxicam, etodolac, nabumetone, sulidac, tolementin, diclofenac, diflunisal, indomethacin, ketoprofen, oxaprozin, and piroxicam.

13. The method of claim 11, wherein the nanosized drug delivery vehicle is a stabilized liposome.

14. The method of claim 13, wherein the liposome has a diameter about 100 nm.

15. The method of claim 13, wherein the stabilized liposome has polyethylene glycol polymers (PEG) attached to the exterior surface of the liposome, and the sTNF-R is chemically linked to an active site on a distal free end of the PEG such that the attached sTNF-R is capable of binding to TNF-a.

16. A method of delivering anti-inflammatory drugs to a site of inflammation, said method comprising providing a therapeutic dosage of a nanosized drug delivery vehicle to a patient suffering inflammation, wherein the nanosized drug delivery vehicle encapsulates at least one anti-inflammatory drug and has a soluble tumor necrosis factor receptor (sTNF-R) attached to an exterior surface of the nanosized drug delivery vehicle.

17. The method of claim 16, wherein the nano sized drug delivery vehicle is injected intraveneously.

18. The method of claim 16, wherein the nano sized drug delivery vehicle is injected subcutaneously.

19. The method of claim 16, wherein the nano sized drug delivery vehicle is injected directly into the site of inflammation.

20. The method of claim 16, wherein the at least one anti-inflammatory drug encapsulated in step a) is selected from the group consisting of cortisone, hydrocortisone, prednisolone, methyl prednisolone, methotrexate, hydroxychloroquine, leflunomide, minocycline, sulfasalazine, colchicine, cyclophosphamide, azathioprine, cyclosporine-A, and d-penicillamine, aspirin, ibuprofen, naproxen, meloxicam, etodolac, nabumetone, sulidac, tolementin, diclofenac, diflunisal, indomethacin, ketoprofen, oxaprozin, and piroxicam.
